(19) United States
(12) Patent Application Publication
(10) Pub. No.: US 2022/0160731 A1
(43) Pub. Date: May 26, 2022

(54) COMPOSITIONS COMPRISING BIOLOGICALLY ACTIVE AGENTS AND BILE SALTS

(71) Applicant: POVIVA CORP., Carson City, NV (US)

(72) Inventors: John Docherty, Port Perry (CA); Christopher Andrew Bunka, Kelowna (CA)

(21) Appl. No.: 17/053,529

(22) PCT Filed: May 19, 2020
(86) PCT No.: PCT/US20/33577

§ 371 Date: Nov. 6, 2020

Related U.S. Application Data
(60) Provisional application No. 62/850,509, filed on May 20, 2019.

(51) Int. Cl. A61K 31/575; A61K 31/05; A61K 31/352; A61K 31/465; A61K 47/44; A61K 47/36

(52) U.S. Cl. CPC A61K 31/575; A61K 31/05; A61K 47/36; A61K 31/465; A61K 47/44; A61K 31/352

(57) ABSTRACT

Disclosed herein are compositions capable of enhanced delivery and absorption of biologically active agents when administered to a subject. Further disclosed are methods for increasing the in vivo absorption of biologically active agents.

### FIELD

Disclosed herein are compositions capable of enhanced delivery and absorption of biologically active agents when administered to a subject. Further disclosed are methods for increasing the in vivo absorption of biologically active agents.

### BRIEF DESCRIPTION OF THE FIGURES

FIG. 1 contrasts the CBD plasma levels achieved with a control (▪) and a disclosed composition (●).

### DETAILED DESCRIPTION

The materials, compounds, compositions, articles, and methods described herein may be understood more readily by reference to the following detailed description of specific aspects of the disclosed subject matter and the Examples included therein.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

The terms “a” and “an” are defined as one or more unless this disclosure explicitly requires otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Any embodiment of any of the disclosed methods or compositions can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Any embodiment of any of the disclosed compounds or methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

The term “delivery matrix” and “base substrate” are used interchangeably throughout the disclosure.

As disclosed herein the terms “bile acid,” “bile salt,” “bile acid/salt,” “bile acids,” “bile salts,” and “bile acids/salts” are, unless otherwise indicated, utilized interchangeably herein. Any reference to a bile acid used herein includes reference to a bile acid or a salt thereof. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted.

Disclosed herein are compositions that can increased the absorption of biologically active ingredients into the blood stream of a subject. For example, the user will have a higher plasma level of the active ingredient when to active is formulated as disclosed herein. This results, therefore, in two opportunities for the formulator and the user. Because more of the active ingredient is absorbed into the blood stream, the formulator can use less active to provide the user with the same benefit or result. In addition, the formulator can provide a higher biological benefit using the traditional amount of active agent.

### COMPOSITIONS

Disclosed herein are compositions comprising:
a) one or more biologically active ingredients;
b) a bioavailability enhancing agent;
c) a base substrate; and
d) one or more bile salts.

Further disclosed herein are compositions comprising:
a) one or more biologically active ingredients;
b) a bioavailability enhancing agent;
c) a base substrate;
d) one or more bile salts; and
e) one or more adjunct ingredients.

### Biologically Active Ingredients

As disclosed herein, the biologically active ingredient is any compound which can elicit a biological response in the subject ingesting the disclosed compositions. Non-limiting examples of biologically active ingredients include cannabinoids, nicotine, non-steroidal anti-inflammatory drugs (NSAIDS), vitamins, and the like.

Cannabinoids

One aspect of the disclosure relates to compositions comprising one or more cannabinoids. As used herein the term “cannabinoid” refers to a compound that acts on the cannabinoid receptor. For example, cannabinoids are ligands to cannabinoid receptors (CB1, CB2) found in the human body (Pertwee (1997) Pharmacol. Ther. 74:129-180). The cannabinoids are typically divided into the following groups: classical cannabinoids; non-classical cannabinoids; aminoalkylindole-derivatives; and eicosanoids (Pertwee (1997) Pharmacol. Ther. 74:129-180). Classical cannabinoids are those that have been isolated from C. sativa L. or their synthetic analogs. Non-classical cannabinoids are bi- or tri-cyclic analogs of tetrahydrocannabinol (THC) (without the pyran ring). Aminoalkylindoles and eicosanoids are substantially different in structure compared to classical and non-classical cannabinoids. The most common natural plant cannabinoids (phytocannabinoids) are cannabidiol (CBD), cannabigerol (CBG), cannabichromene (CBC), and cannabinol (CBN). The most psychoactive cannabinoid is Δ⁹-tetrahydrocannabinol.

Therapeutic use of cannabinoids has been hampered by the psychoactive properties of some compounds (e.g., Dronabinol) as well as their low bioavailability when administered orally. Bioavailability refers to the extent and rate at which the active moiety (drug or metabolite) enters systemic circulation, thereby accessing the site of action. The low bioavailability of orally ingested cannabinoids (from about 6% to 20%; Adams & Martin (1996) Addiction 91: 1585-614; Agurell et al. (1986) Pharmacol. Rev. 38: 21-43; Grotenhermen (2003) Clin. Pharmacokinet. 42: 327-60) has been attributed to their poor dissolution properties and extensive first pass metabolism.

Cannabinoids are a heteromorphic group of chemicals which directly or indirectly activate the body's cannabinoid receptors. There are three main types of cannabinoids: herbal cannabinoids that occur uniquely in the cannabis plant, synthetic cannabinoids that are manufactured, and endogenous cannabinoids that are produced in vivo. Herbal cannabinoids are nearly insoluble in water but soluble in lipids, alcohol, and non-polar organic solvents. These natural cannabinoids are concentrated in a viscous resin that is produced in glandular structures known as trichomes. In addition to cannabinoids, the resin is rich in terpenes, which are largely responsible for the odor of the cannabis plant.

Unlike Δ⁹-tetrahydrocannabinol, which exerts its action by binding to CB1 and CB2, cannabidiol does not bind to these receptors and hence has no psychotropic activity. Instead, cannabidiol indirectly stimulates endogenous cannabinoid signaling by suppressing the enzyme that breaks down anandamide (fatty acid amide hydroxylase, “FAAH”). Cannabidiol also stimulates the release of 2-AG. Cannabidiol has been reported to have immunomodulating and anti-inflammatory properties, to exhibit anticonvulsive, anti-anxiety, and antipsychotic activity, and to function as an efficient neuroprotective antioxidant.

Non-limiting examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, Δ⁹-tetrahydrocannabinolic acid A, delta-9-tetrahydrocannabinolic acid B, Δ⁹-tetrahydrocannabinolic acid-C4, Δ⁹-tetrahydrocannabi-varinic acid, Δ⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9- propyl-2,6-methano-2H-1-benzoxocin-5-methanol-cannabiripsol, trihydroxy- Δ⁹-tetrahydrocannabinol, and cannabinol. Examples of cannabinoids within the context of this disclosure include tetrahydrocannabinol and cannabidiol.

As used herein, the term “tetrahydrocannabinol” (THC) refers to a compound having the following formula:

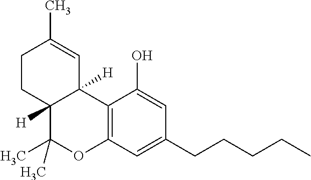 [SMILES: CCCCCc1cc(O)c2c(c1)OC(C)(C)[C@@H]1CCC(C)=C[C@@H]21]

As used herein, the term “cannabidiol” (CBD) refers to a compound having the following formula:

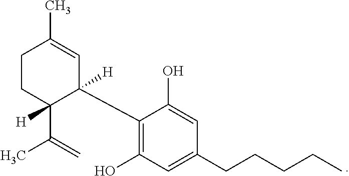 [SMILES: C=C(C)[C@@H]1CCC(C)=C[C@H]1c1c(O)cc(CCCCC)cc1O]

As described herein below, the disclosed compositions can comprise from about 2.5 mg to about 250 mg of a cannabinoid.

Nicotine

The disclosed compositions can comprise an effective amount of nicotine sufficient to satisfy the craving that a subject experiences. The delivery of nicotine via the disclosed compositions is effective for controlling the use of cigarettes, cigars and smokeless tobacco.

For the purposes of the present disclosure “nicotine” includes (S)-3-(1-methylpyrrolidin-2-yl)pyridine, the compound itself, as well as, nicotine mimetics, active metabolites, receptor agonists, and compounds synthesized to aid in smoking cessation.

The disclosed compositions can comprise nicotine in other forms, for example, an acid addition salt, for example, nicotine hydrogen tartrate, nicotine bitartrate dihydrate, nicotine hydrochloride, nicotine dihydrochloride, nicotine sulfate, nicotine citrate, nicotine zinc chloride monohydrate, nicotine salicylate, nicotine oil, and nicotine complexed with cyclodextrin nicotine hydrogen tartrate, nicotine bitartrate dihydrate, nicotine hydrochloride, nicotine dihydrochloride, nicotine sulfate, nicotine citrate, nicotine zinc chloride monohydrate, nicotine salicylate, nicotine oil, or nicotine complexed with cyclodextrin.

The disclosed compositions can also comprises nicotine derivatives, for example, nornicotine, (S)-cotinine, B-nicotyrine, (S)-nicotene-N′-oxide, anabasine, anatabine, myosmine, B-nornicotyrine, 4-(methylamino)-1-(3-pyridyl)-1-butene (metanicotine) cis or trans, N′-methylanabasine, N′-methylanatabine, N′-methylmyosmine, 4-(methylamino)-1-(3-pyridyl)-1-butanone (pseudoxynicotine), 2,3′-Bipyridyl, lobeline, cytisine, nicotine polacrilex, nornicotine, nicotine 1-N-oxide, metanicotine, nicotine imine, nicotine N-glucuronide, N-methylnicotinium, N-n-decylnicotinium, 5′-cyanonicotine, 3,4-dihydrometanicotine, N′-methylnicotinium, N-octanoylnornicotine, 2,3,3a,4,5,9b-hexahydro-1-methyl-1H-pyrrolo(3,2-h)isoquinoline, 5-isothiocyanonicotine, 5-iodonicotine, 5′-hydroxycotinine-N-oxide, homoazanicotine, nicotine monomethiodide, N-4-azido-2-nitrophenylnornicotine, N-methylnornicotinium, nicotinium molybdophosphate resin, N-methyl-N′-oxonicotinium, N′-propylnornicotine, pseudooxynicotine, 4′-methylnicotine, 5-fluoronicotine, K(s-nic)5(Ga2(N,N′-bis-(2,3-dihydroxybenzoyl)-1,4-phenylenediamine)3), 5-methoxynicotine, 1-benzyl-4-phenylnicotinamidinium, 6-n-propylnicotine, SIB1663, 6-hydroxynicotine, N-methyl-nicotine, 6-(2-phenylethyl)nicotine, N′-formylnornicotine, N-n-octylnicotinium, N-(n-oct-3-enyl)nicotinium, N-(n-dec-9-enyl)nicotinium, 5′-acetoxy-N′-nitrosonornicotine, 4-hydroxynicotine, 4-(dimethylphenylsilyl)nicotine, N′-carbomethoxy-nornicotine, or N-methylnicoton.

In addition the nicotine compound can be an agonist having selectivity to the α₇ nicotinic receptor subtype, for example, N-[(2S,35)-2-(pyridin-3-ylmethyl)-1-azabicyclo[2.2.2]oct-3-yl]-1-benzofur-an-2-carboxamide, (5aS,8S,10aR)-5a,6,9,10-Tetrahydro, 7H,11H-8,10a-methanopy-rido[2′,3′:5,6]pyrano[2,3-d]azepine, 1,4-diazabicyclo[3.2.2]nonane-4-carboxylic acid, 4-bromophenyl ester, 3-[(3E)-3-[(2,4-dimethoxyphenyl)methylidene]-5,6-dihydro-4H-pyridin-2-yl]- pyridine, 2-methyl-5-(6-phenyl-pyridazin-3-yl)-octahydro-pyrrolo[3,4-c]pyrrole, (5S)-spiro[1,3-oxazolidine -5,8′-1-azabicyclo[2.2.2]octane]-2-one, N-[(3R)-1-azabicyclo[2.2.2]oct-3-yl]-4-chlorobenzamide, 5-morpholin-4-yl-pentanoic acid (4-pyridin-3-yl-phenyl)-amide, EVP-6124, EVP-4473, TC-6987, and MEM3454.

In another embodiment, the nicotine compound can be an agonist having selectivity to an 42 nicotinic receptor subtype, for example, 7,8,9,10-tetrahydro-6,10-methano-6H-pyrazino(2,3-h)(3) benzazepine, (2S,4E)-5-(5-isopropoxypyridin-3-yl)-N-methylpent-4-en-2-amine, [3-(2(S))-azetidinylmethoxy)pyridine] dihydrochloride, (5aS,8S,10aR)-5a,6,9,10-Tetrahydro, 7H,11H-8,10a-methanopyrido [2′,3′:5,6]pyrano[2,3-d]azepine, A-969933, S35836-1, S35678-1, and 3-(5,6-Dichloro-pyridin-3-yl)-1S,5S-3,6-diazabicyclo[3.2.0]-heptane.

As described herein below, the disclosed compositions can comprise from about 2.5 mg to about 250 mg of nicotine.

Non-Steroidal Anti-inflammatory Drugs (NSAIDS)

The disclosed compositions can comprise from about 2.5 mg to about 250 mg of one or more NSAIDS. Non-limiting examples of NSAIDS include acetylsalicylic acid, ibuprophen, acetaminophen, diclofenac, indomethacin, and piroxicam.

Vitamins

The disclosed compositions can comprise from about 2.5 mg to about 250 mg of one or more lipid soluble vitamins, i.e., vitamin A and vitamin E. Included herein are the carotenoids, for example, retinol, retinal, retinoic acid, α-carotene, β-carotene, γ-carotene and δ-carotene. Also disclosed herein are the vitamin E tocopherols α-tocopherol, β-tocopherol, γ-tocopherol and δ-tocopherol.

The disclosed single dose compositions can comprise any amount of biologically active ingredient from about 2.5 mg to about 250 mg. For example, the disclosed compositions can comprise lower doses of the biologically active ingredients. In one low dose embodiment the compositions comprise from about 2.5 mg to about 10 mg of the active ingredient. In another low dose embodiment the compositions comprise from about 5 mg to about 10 mg of the active ingredient. In a further low dose embodiment the compositions comprise from about 2.5 mg to about 5.0 mg of the active ingredient. In still further low dose embodiment the compositions comprise from about 4 mg to about 8 mg of the active ingredient. In a yet further low dose embodiment the compositions comprise from about 5 mg to about 7.5 mg of the active ingredient. The disclosed compositions can comprise from about 2.5 mg to about 10.0 mg, for example, 2.5 mg, 2.6 mg, 2.7 mg, 2.8 mg, 2.9 mg, 3.0 mg 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, and 10.0 mg,

The disclosed compositions can comprise a higher dose of the biologically active ingredients, for example, from about 25 mg to about 250 mg. In one higher dose embodiment the compositions comprise from about 25 mg to about 100 mg of active ingredient. In another higher dose embodiment the compositions comprise from about 100 mg to about 200 mg of active ingredient. In a further higher dose embodiment the compositions comprise from about 50 mg to about 150 mg of active ingredient. In a still further higher dose embodiment the compositions comprise from about 75 mg to about 125 mg of active ingredient. In a yet further higher dose embodiment the compositions comprise from about 150 mg to about 250 mg of active ingredient.

As such, the disclosed compositions can comprise from about 25 mg to about 250 mg of one or more biologically active ingredients, for example, 25 mg, 26 mg, 27 mg, 28 mg, 29 mg, 30 mg 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102, mg, 103, mg, 104 mg, 105 mg, 106 mg, 107 mg, 108 mg, 109 mg, 120 mg, 121 mg, 122 mg, 123 mg, 124 mg, 12.5 mg, 126 mg, 127 mg, 128 mg, 129 mg, 120 mg, 1 21 mg, 122 mg, 123 mg, 124 mg, 12.5 mg, 126 mg, 127 mg, 128 mg, 129 mg, 130 mg 31 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 1 62 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 190 mg, 191 mg, 192 mg, 193 mg, 194 mg, 195 mg, 196 mg, 197 mg, 198 mg, 199 mg, 200 mg, 201 mg, 202, mg, 203, mg, 204 mg, 205 mg, 206 mg, 207 mg, 208 mg, 209 mg, 210 mg, 212 mg, 212 mg, 213 mg, 214 mg, 215 mg, 216 mg, 217 mg, 218 mg, 219 mg, 220 mg, 2 21 mg, 222 mg, 223 mg, 224 mg, 22.5 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg.

The disclosed compositions can provide a single dose of a disclosed biologically active ingredient based upon the body mass of the subject being treated. Therefore, a single dose of a disclosed biologically active ingredient can range from about 0.5 mg/kg to about 20 mg/kg of the subject's body mass. In one embodiment, the amount of a disclosed biologically active ingredient in a single dose is from about 1 mg/kg to about 8 mg/kg of the subject's body mass. In another embodiment, the amount of a disclosed biologically active ingredient in a single dose is from about 2 mg/kg to about 5 mg/kg of the subject's body mass. In a further embodiment, the amount of a disclosed biologically active ingredient in a single dose is from about 1.5 mg/kg to about 4 mg/kg of the subject's body mass. In a yet further embodiment, the amount of a disclosed biologically active ingredient in a single dose is from about 4 mg/kg to about 10 mg/kg of the subject's body mass. In a still further embodiment, the amount of a disclosed biologically active ingredient in a single dose is from about 5 mg/kg to about8 mg/kg of the subject's body mass. For example, the dose can comprise any amount from about 0.5 mg/kg to about 10 mg/kg on the body mass of the subject being treated. For example, 0.5 mg/kg, 0.6 mg/kg, 0.7 mg/kg, 0.8 mg/ kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 50 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 90 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or 10.0 mg/kg of a subject's body mass.

Actual dosage levels of the biologically active ingredients in the disclosed compositions can vary to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular active ingredient employed, the route of administration, the time of administration, the rate of excretion of the particular biologically active ingredient being employed, the duration of the treatment, other drugs, and/or materials used in combination with the particular active ingredient employed, the age, sex, weight, condition, general health and prior medical history of the patient being treated, and like factors well known in the medical arts.

### Bioavailability Enhancing Agent

The disclosed bioavailability enhancing agent comprises one or more triglycerides. In one aspect the disclosed triglycerides are edible oils. An edible oil is defined herein as an oil that is capable of undergoing de-esterification or hydrolysis in the presence of pancreatic lipase in vivo under normal physiological conditions. Specifically, digestible oils comprise glycerol triesters of C₆-C₂₂ fatty acids.

The disclosed edible oils can have a low percentage of saturated fatty acids, for example, hemp seed oil (7.0%) or a high percentage of saturated fatty acids, for example, coconut oil (82.5%) provide the solid content index is such that the oil is liquid and flowable at temperatures above about 15° C.

In one aspect of the disclosed bioavailability enhancing agents the triglycerides comprise less than or equal to about 5% by weight of free fatty acids, mono-glycerides and di-glycerides. The triglycerides of the disclosed bioavailability enhancing agent are refined, bleached and de-odorized.

Vegetable oils comprise the disclosed triglycerides. These oils are refined in order to remove the non-glyceride impurities that are present in the crude oil. Some of these impurities are naturally present in the seeds or formed during harvesting and storage of seeds or during extraction of crude oil and subsequently during its refining. Oil refining processes for vegetable oils are designed to remove these impurities from the oil or reduce them to a level where their deleterious effects on oil stability are minimal and made suitable for human consumption or for pharmaceutical formulation. Vegetable oil undergoes degradation almost immediately after the seed is crushed. The oil starts to show the sign of primary oxidation as measured by its peroxide value. Under certain circumstances the oil may develop a darker color or higher free fatty acids and eventually an unpleasant odor or viscosity. Gums, phosphatides and mucilaginous substances act as emulsifiers increasing loss of oil and can decompose at processing temperatures. Free fatty acids increase foaming and diminish the storage and formulating properties of the disclosed oils.

Presence of compounds such as phosphatides, free fatty acids, odiferous volatiles, colourant, waxes and metal compounds in oil negatively affect the desired properties for compounding with the disclosed antiviral compounds and storage stability of the refined oil avoids the presence of any unwanted or reactive species being a part of the final composition. Refining processes have, therefore, been developed to remove undesirable compounds such as tocopherols, phenols, sterols and the like.

Chemical refining includes degumming, neutralizing, bleaching, winterizing and de-odorizing stages. The edible oils of the disclosed bioavailability enhancing agents are refined oils that have been winterized to prevent the precipitation of wax.

Disclosed herein are non-limiting examples of edible oils suitable for use in delivering antiviral agents. Plant based oils include borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, soybean oil, sunflower oil, tree nut oil, hydrogenated castor oil, hydrogenated coconut oil, hydrogenated palm oil, hydrogenated soybean oil, hydrogenated vegetable oil, glyceryl trioleate, glyceryl trilinoleate, glyceryl trilinolenate, citrate thisocetyl triglyceride having 10-18 carbon atoms, omega-3 polyunsaturated fatty acid triglyceride containing oil, omega-3 oil, omega-6 oil, and any combination thereof.

In one aspect the edible oils comprise one or more fish oils. Included within fish oil are algal oils. Non-limiting examples of fish oils include herring, sardines, Spanish mackerel, salmon, halibut, tuna, swordfish, tilefish, pollock, cod, catfish, flounder, grouper mahi mahi, orange roughy, red snapper, shark, king mackerel, hoki, and gemfish.

Edible oils having a plurality of non-conjugated di-enes and tri-enes, for example, linoleic and linolenic acids, can by “touch hardened” to increase the amount of mono-olefins present. Touch harden refers to hydrogenation to a point wherein the Iodine value of the triglyceride is lowered to 1-107 or less.

### Base Substrate

The disclosed compositions can comprise a base substrate as a matrix for delivery of the disclosed antiviral agents. Base substrates can include any solid food product. Non-limiting examples of base substrates include meats, fish, fruits, vegetables, dairy products, legumes, pastas, breads, grains, seeds, nuts, spices, and herbs. In addition, the bioavailability enhancing agent and the antiviral agent can be combined into a beverage. Non-limiting examples of beverages includes coffee, tea, milk products and the like.

The disclosed comestibles can include a dry particulate base. For example, a starch such as tapioca starch, corn starch, potato starch, gelatin, dextrin, inulin, cyclodextrin, oxidized starch, starch ester, starch ether, crosslinked starch, alpha starch, octenylsuccinate ester, and processed starch obtained by treating a starch by an acid, heat, or enzyme, or an emulsifier such as gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, and combinations thereof, or a sugar alcohol that can optionally have humectant properties such as ethylene glycol, glycerol, erythritol, threitol, arabitol, xylitol, ribitol, mannitol, sorbitol, galactitol, frucitol, iditol, sucrose, fructose, isomalt, maltitol, lactitol, sorbitol, dextrose or inositol, and combinations thereof.

### Bile Salts

The disclosed compositions comprise one or more bile salts. The bile salts enhance the ability of the disclosed compositions to target the duodenum. Non-limiting examples of bile salts and/or bile acids includes steroid acids (and/or the carboxylate anion thereof) and salts thereof, found in the bile of an animal (e.g., a human), including cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, lithocolate, and the like. Taurocholic acid and/or taurocholate are referred to herein as TCA.

Bile salts are typically conjugated with glycine or taurine. For example, the term “bile acid” as used herein includes cholic acid conjugated with either glycine or taurine: glycocholate and taurocholate, respectively (and salts thereof). Any reference to a bile salt or bile acid used herein includes reference to an identical compound naturally or synthetically prepared.

### Adjunct Ingredients

The disclosed compositions can comprise one or more adjunct ingredients.

### Surfactants

The disclosed compositions can comprise one or more surfactants.

Natural Extract Surfactants

One category of suitable surfactants includes compounds that are extracted from plant material that have surfactant activity. The compositions can comprise from about 0.05% to about 0.5% by weight of one or more natural surfactants. Non-limiting examples include extracts of Gynostemma Pentapphyllum, Panax Ginseng, Sapindus mukorossi, cucumis sativus, Olea Europea, and the like. Also suitable for use are mixtures of extracts having surfactant properties.

Anionic Surfactants

The disclosed compositions can comprise one or more C₁₀-C₁₈ alkyl alkoxy sulfates having the formula:

CH₃ (CH₂)ₓ(OCH₂CH₂)_{y}OSO₃M

wherein the index x is from 9 to 17, y is from 1 to 7 and M is a water soluble cation chosen from ammonium, lithium, sodium, potassium and mixtures thereof. A non-limiting example includes sodium dodecyl diethoxy sulfate having the formula:

CH₃ (CH₂)₁₁(OCH₂CH₂)2OSO₃Na.

Alkyl alkoxy sulfates are also commercially available as a mixture of ethoxylates, for example, sodium laureth sulfate is available as a mixture of ethoxylates, i.e., the index y is from 2 to 4. Other suitable examples include sodium laureth-2 sulfate having an average of 2 ethoxylates and a C₁₂ linear alkyl chain. Sodium laureth-2 is available as Texapon™ N 56 from Cognis Corp. Further examples of alkyl alkoxy sulfates includes sodium laureth-1 sulfate, sodium laureth-3 sulfate, sodium laureth-4 sulfate, sodium myreth-2 sulfate and sodium myreth-3 sulfate.

The disclosed compositions can comprise one or more C₁₀-C₁₈ alkyl alkoxy carboxylates having the formula:

CH₃(CH₂)ₓ(OCH₂CH₂)_{y}CO₂M

wherein the index x is from 9 to 17, y is from 1 to 5 and M is a water soluble cation chosen from ammonium, lithium, sodium, potassium and mixtures thereof. A non-limiting example includes sodium dodecyl diethoxy carboxylate having the formula:

CH₃(CH₂)₁₁(OCH₂CH₂)₂CO₂Na.

Alkyl alkoxy carboxylates are also commercially available as a mixture of ethoxylates, for example, sodium laureth sulfate is available as a mixture of ethoxylates, i.e., the index y is from 2 to 4. Other suitable examples include sodium laureth-2 sulfate having an average of 2 ethoxylates and a C₁₂ linear alkyl chain. Sodium laureth-2 is available as Texapon™ N 56 from Cognis Corp. Further examples of alkyl alkoxy sulfates include sodium laureth-1 sulfate, sodium laureth-3 sulfate, sodium laureth-4 sulfate, sodium myreth-2 sulfate and sodium myreth-3 sulfate.

The disclosed compositions can comprise one or more C₁₀-C₁₈ isethionate esters of alkyl alkoxy carboxylates having the formula:

CH₃(CH₂)ₓ(OCH₂CH₂)_{y}OCH₂C(O)OCH₂CH₂SO₃M

wherein the index x is from 9 to 17, the index y is from 1 to 5 and M is a water soluble cation. Isethionate esters of alkyl alkoxy carboxylates are described in U.S. Pat. No. 5,466,396 the disclosure of which is included herein by reference in its entirety.

The disclosed compositions can comprise one or more C₁₀-C₁₈ alkyl carboxyamides having the formula:

CH₃(CH₂)ₓC(O)NR(CH₂)_{y}CO₂M

wherein R is hydrogen or methyl the index x is from 9 to 17, the index y is froml to 5 and M is a water soluble cation. A non-limiting example of an alkyl carboxyamide suitable for use in the disclosed compositions includes potassium cocoyl glycinate available as AMILITE™ GCK-12 from Ajinomoto. A further example includes compounds wherein R is methyl, for example, sodium cocoyl sarcosinate.

Zwitterionic Surfactants

One category of zwitterionic surfactants relates to C₁₀-C₁₆ alkyl amide betaines having the formula:

CH₃(CH₂)_{w}C(O)NH(CH₂)ᵤN⁺(CH₃)₂(CH₂)ₜCO₂⁻

wherein the index w is from 9 to 15, the index u is from 1 to 5 and the index t is from 1 to 5. Non-limiting examples of betaine surfactants includes {[3-(decanoylamino)ethyl]-(dimethyl)-ammonio}acetate, {[3-(decanoylamino)ethyl](dimethyl)ammonio}-acetate, {[3-(dodecanoyl-amino)ethyl](dimethyl)ammonio}acetate, {[3-(dodecanoylamino)propyl]-(dimethyl)-ammonio}acetate, {[3-(dodecanoylamino)-butyl](dimethyl)ammonio}acetate, {[3-(tetra-decanoylamino)ethyl]dimethyl)-ammonio}acetate, {[3-(tertadecanoylamino)-propyl](dimethyl)ammonio}acetate, {[3-(hexadecanoylamino)ethyl](dimethyl)-ammonio}acetate, and{[3-(hexa-decanoylamino)propyl](dimethyl)ammonio}acetate.

Another category of zwitterionic surfactants relates to C₁₀-C₁₆ alkyl amide sultaines having the formula:

CH₃(CH₂)_{w}C(O)NH(CH₂)ᵤN⁺(CH₃)₂(CH₂)ₜSO₃⁻

wherein the index w is from 9 to 15, the index u is from 1 to 5 and the index t is from 1 to 5. Non-limiting examples of sultaine surfactants includes {[3-(decanoylamino)ethyl]-(dimethyl)-ammonio}methanesulfonate, {[3-(decanoylamino)ethyl](dimethyl)ammonio}-methanesulfonate, {[3-(dodecanoyl-amino)ethyl](dimethyl)ammonio}methanesulfonate, {[3-(dodecanoylamino)-propyl](dimethyl)ammonio}methanesulfonate, {[3-(dodecanoyl-amino)butyl](dimethyl)-ammonio}methanesulfonate, {[3-(tetradecanoylamino)ethyl]-(dimethyl)ammonio}methane-sulfonate, {[3-(tertadecanoylamino)propyl](dimethyl)-ammonio}methanesulfonate, {[3-(hexadecanoylamino)ethyl](dimethyl)ammonio}-methanesulfonate, and{[3-(hexadecanoylamino)propyl](dimethyl)ammonio}-methanesulfonate.

A further category of zwitterionic surfactants relates to C₁₀-C₁₆ alkyl hydroxy sultaines having the formula:

CH₃(CH₂)_{w}N⁺(CH₃)₂CH₂CHOHCH₂SO₃⁻

wherein the index w is from 9 to 15. Non-limiting examples of alkyl hydroxy sultaine surfactants includes 3-[dodecyl(dimethyl)azaniumyl]-2-hydroxypropane-1-sulfonate (lauryl hydroxysultaine), 3-[tetradecyl(dimethyl)azaniumyl]-2-hydroxypropane-1-sulfonate (myristyl hydroxysultaine), (Z)-{dimethyl [3-(octadec9-enamido)propyl] ammonio}-methanesulfonate (oleyl hydroxysultaine), and the like.

Nonionic Surfactants

One category of nonionic surfactants relates to C₈-C₁₈ alkylglycosidyl nonionic surfactant having the formula:

CH₃(CH₂)_{q}O[G]ₚH

wherein G represents a monosaccharide residue chosen from glucose, fructose, mannose, galactose, talose, allose, altrose, idose, arabinose, xylose, lyxose, ribose and mixtures thereof, the index p is from 1 to 4, the index q is from 7 to 17. The following are non-limiting examples of alkyl glucoside surfactants include (2R,3S,4S,5R,6R)-2-(hydroxymethyl)-6-octooxyoxane-3,4,5-triol (octyl glucoside, n-octyl-β-D-glucoside), (2R,3R,4S,5S,6R)-2-decoxy-6-(hydroxymethyl)tetra-hydropyran-3,4,5-triol (decyl glucoside, n-decyl-β-D-glucoside), and (2R,3R,4S,5S,6R)-2-dodecoxy-6-(hydroxymethyl)tetrahydropyran-3,4,5-triol (dodecyl glucoside, lauryl glucoside, n-dodecyl-β-D-glucoside). One example of a suitable admixture of C₈-C₁₆ alkylglycosidyl nonionic surfactants is PLANTACARE™ 818 UP available from Cogins Chemical Co.

A further category of nonionic surfactants relates to polyoxyethylene glycol alkyl ethers having the formula:

RO(CH₂CH₂O)ₙH

wherein R is a linear or branched alkyl group having from 6 to 20 carbon atoms and n is an integer of about 2 to about 20.

On example of suitable ethoxylate alcohol surfactants are the NEODOL™ ethoxylated alcohols from Shell Chemicals. NEODOL™ 23-1 is a surfactant comprising a mixture of R units that are C₁₂ and C₁₃ in length with an average of 1 ethoxy unit. Non-limiting examples of ethoxylated alcohols include NEODOL™ 23-1, NEODOL™ 23-2, NEODOL™ 23-6.5, NEODOL™ 25-3, NEODOL™ 25-5, NEODOL™ 25-7, NEODOL™ 25-9, PLURONIC™ 12R3, and PLURONIC™ 25R2 available from BASF.

A still further category of nonionic surfactants relates to polyoxyethylene glycol alkyl ethers having the formula:

RO(CH₂CH(CH₃)O)ₙH

wherein R is a linear or branched alkyl group having from 6 to 20 carbon atoms and n is an integer of about 2 to about 20.

Another category of nonionic surfactants suitable for use in the disclosed compositions includes polyoxyethylene polyoxypropylene block copolymers known as “poloxamers” having the formula:

HO(CH₂CH₂)_{y1}(CH₂CH₂CH₂O)_{y2}(CH₂CH₂O)_{y3}OH

these are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol. These extracellular desiccants are also well known by the trade name PLURONICS™. These compounds are commonly named with the word Poloxamer followed by a number to indicate the specific co-polymer, for example Poloxamer 407 having two PEG blocks of about 101 units (y¹ and y³ each equal to 101) and a polypropylene block of about 56 units. This category of nonionic surfactant is commercially, for example, under the trade name LUTROL™ F-17 available from BASF.

Another category of adjunct ingredients includes flavor enhancing agents. Non-limiting examples of flavor enhancing agents include vanilla, vanillin, ethyl vanillin, orange oil, lemon oil, peppermint oil, strawberry, raspberry, and mixtures thereof.

Another example of an adjunct ingredient is an emulsifier which provides a homogeneous composition. Non-limiting examples of emulsifiers includes soy and egg lecithin, mono- and diglycerides, polysorbates, carrageenan, and guar gum. In some embodiments one or more of the bioavailability agents can serve a suitable emulsifier.

### COMPOSITIONS

Disclosed herein are compositions that provide increased absorption of a biologically active ingredient when the composition is administered to a subject.

In one aspect the disclosed compositions comprise:
a) one or more biologically active ingredients;
b) a bioavailability enhancing agent;
c) a base substrate; and
d) one or more bile salts.

One embodiment of this aspect includes compositions comprising:
a) one or more cannabinoids;
b) a bioavailability enhancing agent;
c) a base substrate; and
d) one or more bile salts.

One iteration of this embodiment relates to compositions comprising:
a) an effective amount of CBD;
b) a triglyceride;
c) a starch; and
d) an animal bile extract.

A further iteration comprises:
a) from about 0.5% to about 20% by weight of high CBD-content multi-spectrum hemp oil;
b) from about 0.5% to about 20% by weight of sunflower oil having a high oleic acid content;
c) gum Arabic; and
d) from about 2% to about 25% by weight of ox bile extract;
wherein the ratio of component (a) to component (b) is 1:2.

### General Process for Preparing the Disclosed Compositions

One or more biologically active ingredients are combined with a bioavailability enhancing agent and the ingredients are heated and thoroughly admixed to render a homogenous composition wherein the triglycerides and the biologically active ingredients are in intimate contact. A base substrate is added and the ingredients further admixed. One or more of the disclosed bile salts are added and the ingredients admixed. The composition is then subjected to dehydration, lyophilization or other drying methods to remove all water and volatiles resulting in free flowing powder. The composition can then be combined with one or more optional adjunct ingredients. The final powder can be further processed to produce the desired particle size range.

The control composition comprises
a) high CBD-content multi-spectrum hemp oil as the biologically active ingredient;
b) high oleic acid content sunflower oil as the bioavailability enhancing agent; wherein (a) and (b) are present in a 1:1 ratio; and
c) gum Arabic as the base substrate.

This composition comprised 27.89 mg CBD/g of composition

### EXAMPLE 1

A fine powder formulation was prepared according to the General Process. The composition comprised gum Arabic powder as a base substrate, high CBD-content multi-spectrum hemp oil and high oleic acid sunflower oil in a 1:2 ratio is combined with from about 2% to about 25% Ox Bile extract powder. Once combined the composition was determined to have 27.89 mg of CBD per gram of composition (2.8%).

The test composition comprises:
a) high CBD-content multi-spectrum hemp oil as the biologically active ingredient;
b) high oleic acid content sunflower oil as the bioavailability enhancing agent; wherein (a) and (b) are present in a 1:1 ratio;
c) gum Arabic as the base substrate; and
d) Ox Bile extract powder as the bile salt.

This composition comprised 27.89 mg CBD/g of composition.

### In Vivo Testing

The composition of Example I is dissolved in water to provide 25 mg/kg of CBD in the dosing solution. The control composition and the composition of Example I are orally dosed to the subject animals. Following PO dosing of the Example I formulation at 25 mg/kg of CBD, maximum plasma concentrations (average of 235±111 ng/mL) of CBD were observed between 30 minutes and 1 hour post dosing. The average half-life after oral dosing could not be determined either because the terminal elimination phase was not observed or due to a lack of quantifiable data points trailing the Cₘₐₓ. The average total exposure for CBD was 135±63.7 hr*ng/mL and based on the dose normalized AUCₗₐₛₜ was 5.39±2.55 hr*kg*ng/mL/mg. After PO administration, the average total amount excreted in urine and feces over a 24 hour period was 0.0869 μg (<0.01% of the unchanged dose) and 1004 (13.8% of the unchanged dose), respectively. The average brain tissue concentrations observed at 8 hours and 24 hours were 275±155 ng/g and 6.21±2.00 ng/g, respectively.

By comparison, PO dosing of the Standard Formulation demonstrated maximum plasma concentrations (average of 112±46.6 ng/mL) of CBD that were observed between 30 minutes and 1 hour post dosing. The average half-life after oral dosing could not be determined because the terminal elimination phase was not observed or due to a lack of quantifiable data points trailing the Cₘₐₓ. The average total exposure for CBD was 64.6±23.6 hr*ng/mL and based on the dose normalized AUCₗₐₛₜ was 2.58±0.946 hr*kg*ng/mL/mg. After PO administration, the average total amount excreted in urine and feces over a 24 hour period was 0.0677 μg (<0.01% of the unchanged dose) and 968 (13.5% of the unchanged dose), respectively. The average brain tissue concentrations observed at 8 hours and 24 hours were 46.8±12.3 ng/g and 2.49±0.804 ng/g, respectively.

These data indicated significant differences between the two formulations comparing Cmax achieved with the bile salt formulation (235±111 ng/mL) to that achieved with the standard formulation (112±46.6 ng/mL) (p<0.0001), AUC achieved with the bile salt formulation (135±63.7 hr·ng/mL) to that achieved with the standard formulation (64.6±23.6 hr·ng/mL) (p<0.0001) and also brain levels achieved with the bile salt formulation of 275±155 ng/g at 8 hrs and 6.21±2.00 ng/g at 24 hrs relative to those achieved with the standard formulation of 46.8±12.3 ng/g and 2.49±0.804 ng/g respectively (both p<0.0001).

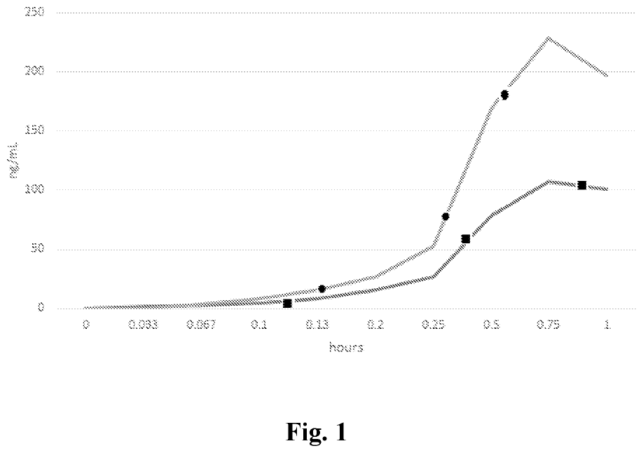

The details of the in vivo testing are summarized in FIG. 1. The control sample is indicated by (▪), and the results for Example I is indicated by (●). As seen in FIG. 1, the plasma levels for the disclosed bile salt containing composition achieved far superior plasma levels.

### METHODS OF USE

The disclosed compositions can be used as a method for delivering a biologically active ingredient to the brain of a subject when the disclosed composition is administered to the subject. As evidenced by the increased concentration of a biologically active ingredient, i.e., cannabinoid, in the brains of test animals, disclosed herein is a method for increasing the average concentration of a biologically active ingredient in the brain tissue of a subject, comprising administering to a subject a composition, comprising:
a) one or more biologically active ingredients;
b) a bioavailability enhancing agent;
c) a base substrate; and
d) one or more bile salts.

The amount of biologically active ingredient that reaches the brain is enhanced over other methods which attempt to deliver an active ingredient across the blood/brain barrier.

In a further aspect of the disclosed methods for using the disclosed compositions, disclosed herein is a method for improving the delivery of a CNS biologically active ingredient to the brain tissue of a subject, comprising administering to a subject a composition, comprising:
a) one or more biologically active ingredients;
b) a bioavailability enhancing agent;
c) a base substrate; and
d) one or more bile salts.

Further disclosed herein is a method for increasing the average concentration of a biologically active ingredient in the blood stream of a subject, comprising administering to a subject a composition, comprising:
a) one or more biologically active ingredients;
b) a bioavailability enhancing agent;
c) a base substrate; and
d) one or more bile salts.

The amount of biologically active ingredient that reaches the brain is enhanced over other methods which attempt to deliver an active ingredient across the blood/brain barrier because the concentration of the active ingredient in the blood stream is much higher when delivered by the disclosed compositions.

In a further aspect of the disclosed methods for using the disclosed compositions, disclosed herein is a method for improving the delivery of a CNS biologically active ingredient to the blood stream of a subject, comprising administering to a subject a composition, comprising:
a) one or more biologically active ingredients;
b) a bioavailability enhancing agent;
c) a base substrate; and
d) one or more bile salts.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

1. A composition comprising:
a) one or more biologically active ingredients;
b) a bioavailability enhancing agent;
c) a base substrate; and
d) one or more bile salts.

2. The composition according to claim 1, wherein the one or more biologically active ingredients is a cannabinoid.

3. The composition according to claim 1, wherein the cannabinoid is chosen from tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C₄, cannabidivarinic acid, cannabidiorcol, Δ⁹-tetrahydrocannabinolic acid A, delta-9-tetrahydrocannabinolic acid B, Δ⁹-tetrahydrocannabinolic acid-C₄, Δ⁹-tetrahydrocannabi-varinic acid, 4⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9-propyl-2,6-methano-2H-1-benzoxocin-5-methanol-cannabiripsol, trihydroxy-Δ⁹-tetrahydrocannabinol, and cannabinol.

4. The composition according to claim 3, wherein the cannabinoid is tetrahydrocannabinol or cnnabidiol.

5. (canceled)

6. The composition according to claim 1, wherein the one or more biologically active ingredients is nicotine.

7. The composition according to claim 1, wherein the one or more biologically active ingredients is a nicotine mimetic, an active nicotine metabolite, an α₇ nicotinic receptor subtype agonist, an α₄Δ₃₂ nicotinic receptor subtype agonist, or compound synthesized to aid in smoking cessation.

8-10. (canceled)

11. The composition according to claim 1 any one of claims 1 to 10, comprising from about 2.5 mg to about 250 mg of the one or more biologically active ingredients.

12. The composition according to claim 1, wherein the bioavailability enhancing agent is an edible oil chosen from borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil, lanolin oil, linseed oil, mink oil, mustard seed oil, olive oil, palm oil, palm kernel oil, rapeseed oil, safflower oil, sesame oil, shark liver oil, soybean oil, sunflower oil, tree nut oil, hydrogenated castor oil, hydrogenated coconut oil, hydrogenated palm oil, hydrogenated soybean oil, hydrogenated vegetable oil, glyceryl trioleate, glyceryl trilinoleate, glyceryl trilinolenate, citrate thisocetyl triglyceride having 10-18 carbon atoms, omega-3 polyunsaturated fatty acid triglyceride containing oil, omega-3 oil, omega-6 oil, or mixtures thereof.

13. (canceled)

14. The composition according to claim 1, wherein the base substrate is a food product chosen from meats, fish, fruits, vegetables, dairy products, legumes, pastas, breads, grains, seeds, nuts, spices, and herbs.

15. The composition according to claim 1, wherein the base substrate is a dehydrated beverage chosen from coffee, tea, or milk products.

16. The composition according to claim 1, wherein the base substrate is tapioca starch, corn starch, potato starch, gelatin, dextrin, inulin, cyclodextrin, oxidized starch, starch ester, starch ether, crosslinked starch, alpha starch, octenylsuccinate ester, and processed starch obtained by treating a starch by an acid, heat, or enzyme, or an emulsifier such as gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, ethylene glycol, glycerol, erythritol, threitol, arabitol, xylitol, ribitol, mannitol, sorbitol, galactitol, frucitol, iditol, sucrose, fructose, isomalt, maltitol, lactitol, sorbitol, dextrose or inositol, and combinations thereof.

17. The composition according to claim 1, wherein the bile salts are chosen from anions of steroid bile acids cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, or lithocolate.

18. The composition according to claim 17, wherein the bile salts are oxen or bovine bile salts.

19. The composition according to claim 1, further comprising a surfactant or an emulsifier.

20. A composition comprising:
a) from about 0.5% to about 20% by weight of a biologically active ingredient;
b) from about 0.5% to about 20% by weight of sunflower oil having a high oleic acid content;
c) gum Arabic; and
d) from about 2% to about 25% by weight of ox bile extract;
wherein the ratio of component (a) to component (b) is 1:2.

21. The composition according to claim 20, wherein the biologically active ingredient is a cannabinoid.

22. The composition according to claim 20, wherein the biologically active ingredient is a high CBD-content multi-spectrum hemp oil.

23. The composition according to claim 20, wherein the biologically active ingredient is nicotine.

24-25. (canceled)

26. A method for increasing the average concentration of a biologically active ingredient in the brain tissue of a subject, comprising administering to a subject a composition, comprising:
a) one or more biologically active ingredients;
b) a bioavailability enhancing agent;
c) a base substrate; and
d) one or more bile salts wherein the bile salts are chosen from anions of steroid bile acids cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, or lithocolate.

27. (canceled)

28. The method according to claim 26, comprising:
a) from about 0.5% to about 20% by weight of a biologically active ingredient chosen from a cannabinoid, high CBD-content multi-spectrum hemp oil, nicotine a nicotine mimetic, an active nicotine metabolite, an a7 nicotinic receptor subtype agonist, an α₄β₂ nicotinic receptor subtype agonist, or compound synthesized to aid in smoking cessation;
b) from about 0.5% to about 20% by weight of sunflower oil having a high oleic acid content;
c) gum Arabic; and
d) from about 2% to about 25% by weight of oxen or bovine bile extract; and
wherein the ratio of component (a) to component (b) is 1:2.

29-51. (canceled)
